(19) United States
(12) Patent Application Publication
(10) Pub. No.: US 2023/0035981 A1
(43) Pub. Date: Feb. 2, 2023

(54) INTELLIGENT DETECTION OF A USER'S STATE AND AUTOMATICALLY PERFORMING AN OPERATION BASED ON THE USER'S STATE

(71) Applicant: Avaya Management L.P., Durham, NC (US)

(72) Inventors: Amit Mishra, Broomfield, CO (US); Michael Sean Siemsen, Thornton, CO (US); Mohammad Ismail Al-Taraireh, Plainfield, IL (US); Rebecca M. Richardson, Boulder, CO (US)

(21) Appl. No.: 17/387,352

(22) Filed: Jul. 28, 2021

(51) Int. Cl. G16H 40/67; G16H 40/63

(52) U.S. Cl. CPC G16H 40/67; G16H 40/63

(57) ABSTRACT

Systems, methods, and software to provide intelligent detection of a user's state and automatically establishing a communication session based on the user's state. In one embodiment, one or more algorithms correlate user data and medical device data associated with a user to determine a state of the user. The system and method also determines an operation to perform based on the determined state of the user. The system and method further automatically initiates the determined operation.

### COPYRIGHT NOTICE

A portion of the disclosure of this patent document contains material that is subject to copyright protection. The copyright owner has not objected to the facsimile reproduction by anyone of the patent document or the patent disclosure as it appears in the Patent and Trademark Office patent files or records, but otherwise reserves all copyright rights whatsoever.

### FIELD OF THE DISCLOSURE

The present disclosure relates generally to systems and methods for communications and particularly to intelligently detecting a user's state and determining an operation to perform based on the determined user's state.

### BACKGROUND

Telemedicine refers to the practice of caring for patients remotely when a provider and patient are not physically present with each other. Modern technology has enabled doctors to consult patients by using HIPAA compliant video-conferencing tools. In the medical industry as of today, a patient or healthcare provider needs to manually initiate communication. Similarly, a healthcare provider needs to manually obtain patient information about the patient's condition (e.g., vitals).

### SUMMARY

Systems and methods disclosed herein provide a way to intelligently detect a user's state and perform an operation based on the determined user's state. For example, the system may detect that the user is in distress requiring immediate attention (e.g., a video call with a provider, dispatching emergency services, etc.). The systems and methods disclosed herein provide patients and healthcare providers a way to communicate that may be automatically initiated based on the patient's condition. For example, if the system intelligently (e.g., using machine-learning, artificial intelligence, etc.) detects the user requires assistance, the system and method automatically connects the user with another user. For example, the user may be connected to a nurse and/or doctor depending on the assistance required. The system and method also allows providers to receive patient information and monitor the patient's condition via continuously monitoring the patient's vitals (e.g., via connected medical devices/peripherals). Advantageously, the system and method disclosed herein automates the monitoring of the patient's condition/vitals, which may be particularly helpful when the patient is in a different location from the provider. This information may be stored to the patient's medical records.

Medical devices, which may be included in a monitoring device, and/or connected to a monitoring device may detect problems in patients and can send notification or perform other operations. A camera or other image capturing device may also be included in the monitoring device, the video/images may be analyzed to identify various emotions using multiple face types and facial expressions through many users of various ages. User and medical device data may be used to train (e.g., machine-learning) the system to identify the facial expressions for the cases of comfortable/no pain, low pain, high pain, anxiousness, distressed, etc. A microphone or other audio capturing device may also be included in the monitoring device. The microphone may be used to capture requests for assistance. The audio may be further used to determine if the user requires assistance (e.g., based on the tone/volume/pitch of voice, audio indicating pain, etc.). This information combined with other data (e.g., medical device data) may then be used to determine a state of the user/issue, and an operation to perform based on the determined state (e.g., automatically notify the doctors/nurses to setup a live call/scheduled call based on the urgency detected on the patient condition).

In some embodiments, user data (audio, video, image data, etc.) may be used to perform a general analysis for emotional recognition to make a determination regarding the patient's mood/condition, which may then be combined with their vital information (e.g., blood pressure, body temperature, pulse rate, respiratory rate, etc.) to determine if the user requires assistance and what type of assistance is required. One or more algorithms can also take information from some other sources (e.g., medical records, social media, device location, etc.) in case emergency services (e.g., police, ambulance, etc.) need to be notified/dispatched. The system may be voice/gesture activated.

The monitoring device may be available in all patient care locations (e.g., in a hospital room, remotely in the patient's home, etc.). The monitoring device may interact with a central solution (e.g., one or more servers) which receives all the data (e.g., audio (NLP processor), video, medical device, etc.) for automatic analysis of the patient's state of health. The monitoring device may also gather the required medical device data (e.g., patient vitals) and transfer the same to the servers. The server correlates the user data (e.g., audio/video) and the medical device data (e.g., patient's vitals), to make a determination of the patient's state (comfortable/neutral, in pain, distressed, etc.). The server uses the determined state to decide on an operation to perform (e.g., send a page/text to the provider along with all details/vitals, setup a real-time communication session (e.g., audio/video call) with the provider for further analysis of the patient's condition/issue(s) along with a text/email with all details/vitals, send a text/email to all related medical staff showing all details/vitals. One or more algorithms may weigh the medical device data more than the user data. In other words, audio/NLP analysis and facial recognition analysis may be used to make a confidence determination in recommending a course of action.

These and other needs are addressed by the various embodiments and aspects presented herein. The embodiments provide a number of advantages depending on the particular configuration.

In one embodiment, a system is provided to achieve an intelligent detection of a user's state, determining an operation to perform based on the determined user state, and automatically performing the determined operation, which may be performed by a microprocessor(s) (herein, “processor”) executing functions or modules, which may include one or more of:

#1. Visual Analysis and Processing module: for Intelligent Video and Image processing through an Artificial Intelligence (AI) driven system. In one embodiment, the processor executes a module responsible for analyzing video data received from a user device to determine a mood of the user (e.g., neutral, happy, distressed). The analysis may comprise making determinations based on the facial features of the user. Additionally, or alternatively, the analysis may further comprise determining body positioning and/or body movement.

#2. Natural Language Processing module: in one embodiment, the processor executes a module used to support the Visual Analysis and Processing module #1 described above, and audio analysis and processing module #3 described below. Here, the natural language processing module will process and analyze audio data in real time to determine context. For example, the captured audio may indicate a wake word to trigger monitoring or that the following audio is an instruction.

Once a particular word is identified, such as from use of the wake word, natural language processing (NLP) may be utilized to determine the context of the sentence and whether the sentence is an instruction to connect to the provider. In another example, the audio may indicate the user is in distress/need of assistance. This determination may be further used to strengthen the fact that the user requires assistance.

#3. Audio Analysis and Processing module: in one embodiment, the processor of the server executes a module to receive audio data from the user device. The audio data may be analyzed for audio characteristics such as intensity/loudness, pitch, tone, etc. The audio data is analyzed, preferably in real-time with other data, such as from the video analysis and processing module and/or the natural language processing module. Other data, such as medical device data may be used to make the determination that the user requires assistance.

#4. Confidence module: in one embodiment, the processor executes a module to execute an action upon receiving a confidence score associated with the determined operation. The module can proactively take actions, based on the confidence score, before, or to avoid, any manual intervention, including upon determining the user requires assistance:

Very low confidence score: Take no action.

Low confidence score: Trigger the presentation of a visual indicator.

Medium: Trigger an audible announcement.

High: Automatically perform determined operation (e.g., establish a communication session, dispatch emergency services), as permitted based on legal considerations.

When a determination is made that the user requires assistance, the specific level of confidence may result in a particular action, including:

Very low confidence score: Take no action.

Low confidence score: Trigger the presentation of a visual and/or audible cue.

Medium: Trigger an audio/text message for additional information.

High: Automatically perform determined operation (e.g., establish a communication session, dispatch emergency services).

In addition to automatically determining the threshold confidence score, the user/provider, or other administrator may configure the threshold values and/or disable continuous monitoring. It may be necessary or beneficial to warn users that they are being monitored, but that such monitoring is solely for the determination of whether the user requires assistance, such as in accordance with the law/legal rules imposed by the local countries/geographies in which the present disclosure will be used.

The data gathered as described above, may then be used to train one or more Machine Learning (ML) models. To reduce false positives, filtering may be performed, such as to exclude redundant or otherwise unusable data. This data is used in subsequent determinations/monitoring.

In another embodiment, with training data and the incoming real time stream (e.g., video and audio data) from an endpoint, the confidence module assigns a confidence score reflecting the confidence that the participant has an issue/requires assistance. In response to the confidence score being above a previously determined threshold, an operation may be automatically performed (e.g., automatically establishing a communication session.)

#5. Alerting module in one embodiment, the processor executes a module to send a notification to an endpoint regarding the user's state/issue. The alert/notification may comprise at least one of: a textual, a visual, and/or an audible alert.

Additionally, the system may include a registration and identification module: in one embodiment, when the processor of the server or system registers the user along with the associated user device, the server is allowed to associate the data (e.g., audio data, video data, user data, user's vital data) arriving at the server with the particular user device. As described herein, at least one processor of the server may execute components/modules in order to determine if a communication session should be automatically established or other action (e.g., dispatch emergency services) should be taken in response to determining the state of the user.

The embodiments herein provide for the analyzing the participants' contributed audio and/or video using NLP/Artificial Intelligence (AI), which may also include machine learning, deep learning, or other machine intelligence and voice recognition techniques to make a determination that the user requires assistance, and automatically take appropriate action before any manual intervention is required.

Various embodiments and aspects of the embodiments are disclosed, including:

In one embodiment, a device is disclosed. The device comprising:
a network interface to a network;
a storage component comprising a non-transitory storage device;
a processor, comprising at least one microprocessor; and wherein the processor, upon accessing machine-executable instructions, cause the processor to: receive user data associated with a user;
receive medical device data associated with the user;
correlate the user data and the medical device data to determine a state of the user;
determine an operation based on the determined state of the user; and automatically initiate the determined operation.

In one embodiment, a method to intelligently detect a user's state and automatically establish a communication session based on the user's state is disclosed. The method comprising:
receiving user data associated with a user;
receiving medical device data associated with the user;
correlating the user data and the medical device data to determine the state of the user;
determining an operation based on the determined state of the user; and
automatically initiating the determined operation.

In another embodiment, a non-transitory, computer-readable medium comprising a set of instructions stored therein which, when executed by a processor, cause the processor to:
receive user data associated with a user;
receive medical device data associated with the user;
correlate the user data and the medical device data to determine a state of the user;
determine an operation based on the determined state of the user; and
automatically initiate the determined operation.

Aspects of any one or more of the foregoing embodiments include user data comprising at least one of: facial expression data, positioning data, movement data, and/or mood data.

Aspects of any one or more of the foregoing embodiments, wherein the user data is received via a microphone and/or a camera of a user device associated with the user.

Aspects of any one or more of the foregoing embodiments, wherein the medical device data comprises at least one of: heart rate, pulse rate, body temperature, respiratory rate, and/or blood pressure.

Aspects of any one or more of the foregoing embodiments, wherein the operation comprises a text message to another user.

Aspects of any one or more of the foregoing embodiments, wherein the operation comprises an audio call to another user.

Aspects of any one or more of the foregoing embodiments, wherein the operation comprises a video call to another user.

Aspects of any one or more of the foregoing embodiments, wherein the operation comprises dispatching emergency services to a location of the user.

Aspects of any one or more of the foregoing embodiments wherein the state of the user comprises one of: neutral, happy, or distressed.

The phrases “at least one,” “one or more,” “or,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B, and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.), or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium that, when read by a microprocessor, causes the microprocessor to execute the instructions encoded therein.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible, non-transitory medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

The term “means” as used herein shall be given its broadest possible interpretation in accordance with 35 U.S.C., Section 112(f) and/or Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The preceding is a simplified summary of the present disclosure to provide an understanding of some aspects of the present disclosure. This summary is neither an extensive nor exhaustive overview of the present disclosure and its various embodiments. It is intended neither to identify key or critical elements of the present disclosure nor to delineate the scope of the present disclosure but to present selected concepts of the present disclosure in a simplified form as an introduction to the more detailed description presented below. As will be appreciated, other embodiments of the present disclosure are possible utilizing, alone or in combination, one or more of the features set forth above or described in detail below. Also, while the disclosure is presented in terms of exemplary embodiments, it should be appreciated that an individual aspect of the disclosure can be separately claimed.

### BRIEF DESCRIPTION OF THE DRAWINGS

The present disclosure is described in conjunction with the appended figures:

FIG. 1 depicts a first system in accordance with embodiments of the present disclosure;

FIG. 2 depicts an example input/output to the system in accordance with embodiments of the present disclosure;

FIGS. 3A-3C depict a second system in accordance with embodiments of the present disclosure;

FIG. 4 depicts a first process in accordance with embodiments of the present disclosure;

FIG. 5 depicts a device in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

Any reference in the description comprising an element number, without a sub-element identifier when the sub-element identifier exists in the figures, when used in the plural, is intended to reference any two or more elements with a like element number. When such a reference is made in the singular form, it is intended to reference one of the elements with the like element number without limitation to a specific one of the elements. Any explicit usage herein to the contrary or providing further qualification or identification shall take precedence.

The exemplary systems and methods of this disclosure will also be described in relation to analysis software, modules, and associated analysis hardware. However, to avoid unnecessarily obscuring the present disclosure, the following description omits well-known structures, components, and devices, which may be omitted from or shown in a simplified form in the figures or otherwise summarized.

For purposes of explanation, numerous details are set forth in order to provide a thorough understanding of the present disclosure. It should be appreciated, however, that the present disclosure may be practiced in a variety of ways beyond the specific details set forth herein.

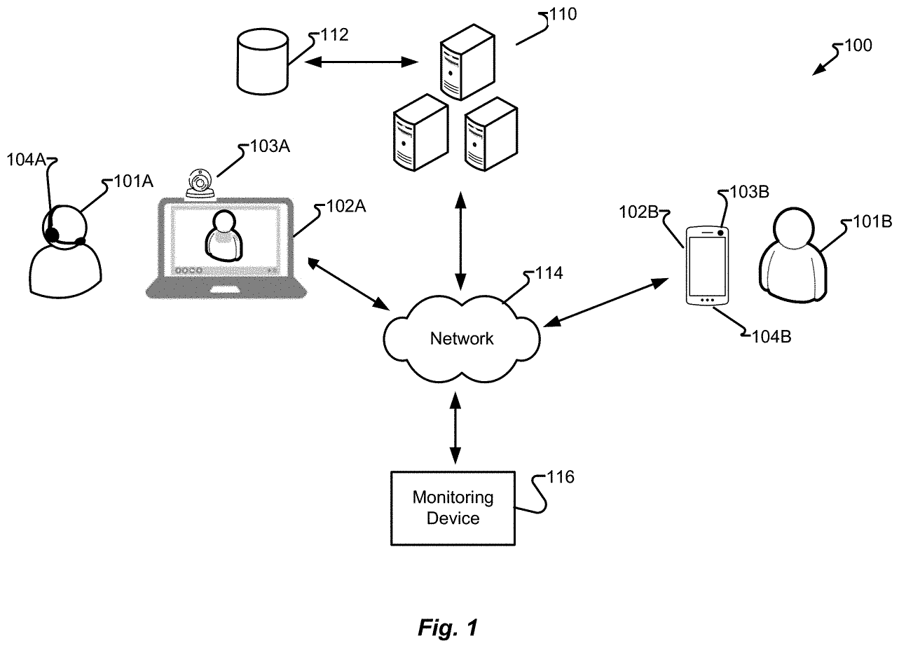

FIG. 1 depicts a system 100 in accordance with embodiments of the present disclosure. In one embodiment, the system 100 includes a user device 102A (including a camera 103A and a headphone/microphone 104A) associated with a user 101A (e.g., provider); a user device 102B (including a camera 103B and a speaker/microphone 104B) associated with a user 101B (e.g., customer/patient); one or more servers 110, a database 112, a network 114, and a monitoring device 116. Although, one user 101A (provider) and one user 101B (patient) are shown, it is understood that there may be one or more users 101A (providers) and one or more users 101B (patients). For example, multiple providers may communicate with one user 101B. Conversely, one user 101A may monitor multiple users 101B.

The system 100 may intelligently detect a state associated with one or both of the user 101A and the user 101B, determine an operation to perform based on the determined state, and automatically perform the determined operation. In some embodiments, the determined operation may be establishing a communication session (the type of communication (e.g., text, audio, video, etc.) may be determined based on the urgency of the condition of one or both of the user 101A and the user 101B, issue afflicting the user, etc.). In one embodiment, the monitoring device 116 collects user data (e.g., audio/video data). In another embodiment, the user data may be sent from the user device 102B to the monitoring device 116. Additionally, or alternatively, the monitoring device 116 may detect utterance of a key/wake word that indicates the following audio should be captured. In some embodiments, the monitoring device 116 may also function to record audio (e.g., treatment notes, doctor orders, etc.) which may then be transcribed or otherwise stored in the medical record for a patient. Use of the wake word/gesture is not required, and the monitoring device 116 may continuously monitor for audio/video. Additionally, the word/instruction may trigger the monitoring device 116 to mute or otherwise stop listening/monitoring. In one embodiment, the server 110 correlates the user data and medical device data (e.g., received from the monitoring device 116) associated with the user (e.g., the user 101A/101B) to determine the state associated with one or both of the users 101A/101B. The server 110 may also determine an operation to perform based on the determined state of the users 101A/101B. The server 110 may further automatically initiate the determined operation (e.g., an audio/video call via the monitoring device 116 or the user device 102), wherein audio, video, documents, co-browsing, and/or other media, is exchanged. The user data may comprise an audio portion in the form of speech. In addition to the audio portion, the user data may comprise a video portion/images. In some embodiments, the monitoring device 116 may perform some or all of the functions performed by the server 110.

The monitoring device 116 converts encoded audio and video signals and transmits the same via the network 114 to the server 110. Optionally, one or more of user devices 102A/102B may similarly transmit encoded audio and video signals to the server 110. Each of the user devices 102 may include the camera 103A/103B to capture images/video to determine facial expression, mood, body position, movement, etc., and a microphone 104A/104B to capture mechanical wave energy (e.g., sound), and converts the sound and images into electrical signals which may be further converted to data packets for transport via the network 114.

The user devices 102A/102B may be embodied as, for example, a laptop with an attached microphone, and an attached camera; a smart phone that includes a camera, and a speaker/microphone; a personal computer with a headset/microphone connected wired or wirelessly, and a camera connected wired or wirelessly; and a video phone that includes a camera, and speaker/microphone. It should be appreciated by those of ordinary skill in the art that other microphones may be utilized as the microphone 104, such as a handset of a telephone, which may be a wired (analog or digital) or wireless (e.g., cellular, WIFI, two-way radio, etc.) to the network 114. Similarly, the user device 102 may be embodied as any telecommunications device operable conduct the required communication session (e.g., text, audio/video call, etc.).

It should be appreciated that the number of users illustrated by the users 101A/101B is non-limiting and may comprise any number of two or more users. As will be discussed more completely with respect to the embodiments that follow, the user data is used to make a determination regarding the state of the user 101A/101B.

The server 110 may have or utilize the database 112 as a non-transitory repository of data accessible to at least one microprocessor (or, more simply, “processor”) of the server 110. The server 110 may be a stand-alone component or co-embodied with other components, such as to manage communications and/or other administrative and/or connectivity features. The server 110 may comprise or access, telephony or other communication equipment (e.g., switches, hubs, routers, etc.) in order to facilitate establishing a communication session, dispatching emergency services, etc. and receiving user data from any of the user devices 102. In another embodiment, the server 110 and/or the database 112 may be embodied as one device.

A much richer experience may be provided to the patient if a communication session may be automatically established when it is detected the patient has an issue/requires assistance. Additionally, collection of patient vitals may be more efficiently gathered in an automated manner. After intelligently detecting the state of the user 101A/101B, the server 110 may determine an operation to perform based on the determined state, the server 110 may also automatically and intelligently take action to perform the determined operation without requiring manual intervention from the user 101A/101B. In some examples, the server 110 may utilize technologies, such as Artificial Intelligence, especially Deep Learning, Image Recognition/facial recognition, and Natural Language Processing to intelligently detect the state of the user 101A/101B.

In some embodiments, an AI Driven Facial Movement Recognition and Analysis module might employ one or more AI Vision libraries which will be trained with numerous samples of human facial structure and facial characteristics in order for the module to recognize different parts of any newly provided facial image and identify the movements of the different facial portions in that image. An artificial neural network may be used to achieve this. NLP may also be based on Machine Learning and an NLP module will also be sufficiently trained, in some cases with the language/terminology of a particular domain in which the conference system will be used. These components may also be services hosted in the cloud as provided by 3rd party cloud service providers.

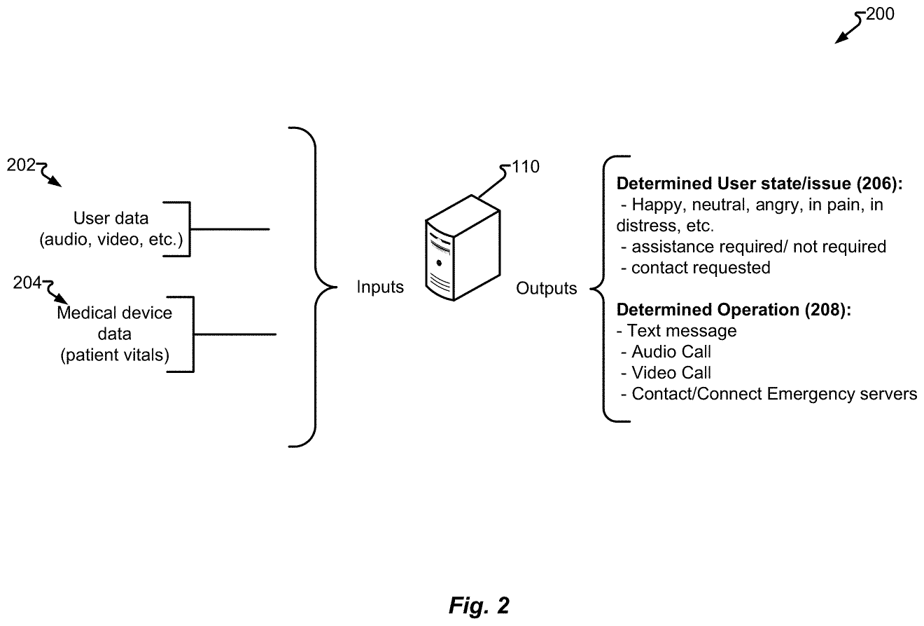

FIG. 2 depicts an example algorithm 200 in accordance with embodiments of the present disclosure. It should be appreciated that the nature of this paper necessitates that spoken content and other sounds, which may be embodied as sound waves or as encoded electrical signals or data packets, be represented as text. This representation using text should not be confused with actual text (e.g., text chat, Short Message Service (SMS), email, etc.). In some embodiments, the algorithm 200 may comprise additional algorithms.

In one embodiment, the server 110 receives user data 202 comprising audio and video content transmitted by user devices 102A/102B and/or monitoring device 116. For example, video data may indicate the patient is distressed. Audio data may indicate the patient has spoken a wake word (depending on the mode). For example, a wake word may be used after the monitoring device 116 has been placed in a sleep mode. In some embodiments, the default mode for the monitoring device 116 is to be constantly monitoring (e.g., audio and/or video) and no wake word/gesture is required. Similar to a wake word, the monitoring device 116 may be muted/put into sleep mode using a keyword/key phrase, gesture, or button. These settings may be configurable, for example the monitoring device 116 may be set to monitor during certain hours, or when the user 101A/101B is alone, etc. The server 110 also receives medical device data 204 (e.g., patient vitals) transmitted by the user device 102 and/or the monitoring device 116. The server 110 correlates the received user data 202 and the medical device data 204 to determine a state/issue 206 of the user (e.g., the user 101A/101B) and determine an operation 208 to perform based on the determined state/issue 206 of the user 101A/101B. For example, if the user/patient is in pain, the operation 208 may be to initiate a video call with a provider (e.g., the user 101A). In another example, if the user 101A/101B or the patient is comfortable/neutral, the operation 208 may be to text/email the provider details regarding the user data 202 and/or the patient vitals 204. The patient's vitals 204 may be sent to the provider periodically similar to a nurse doing patient rounds. In yet another example, if the user 101A/101B or the patient is distressed, the operation may be to dispatch emergency services (e.g., an ambulance) to the user's 101A/101B location.

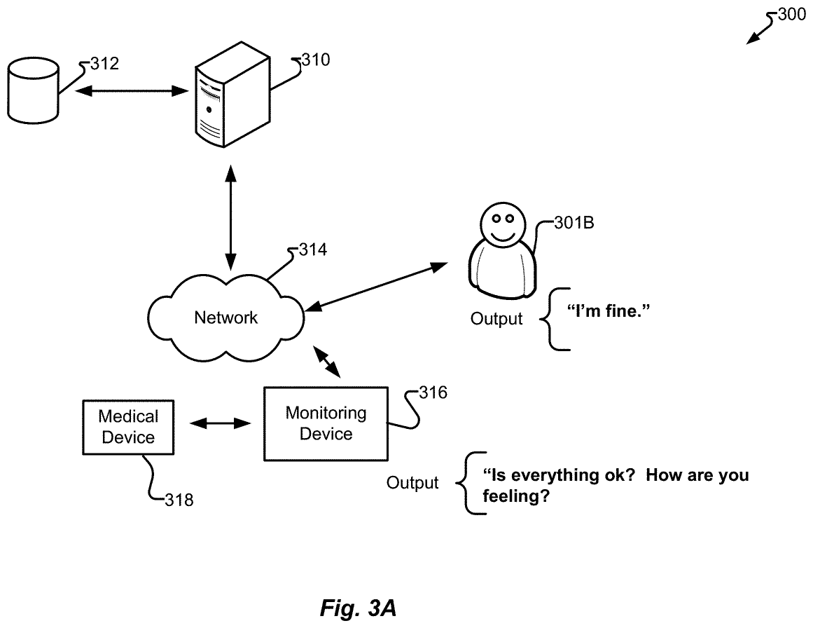

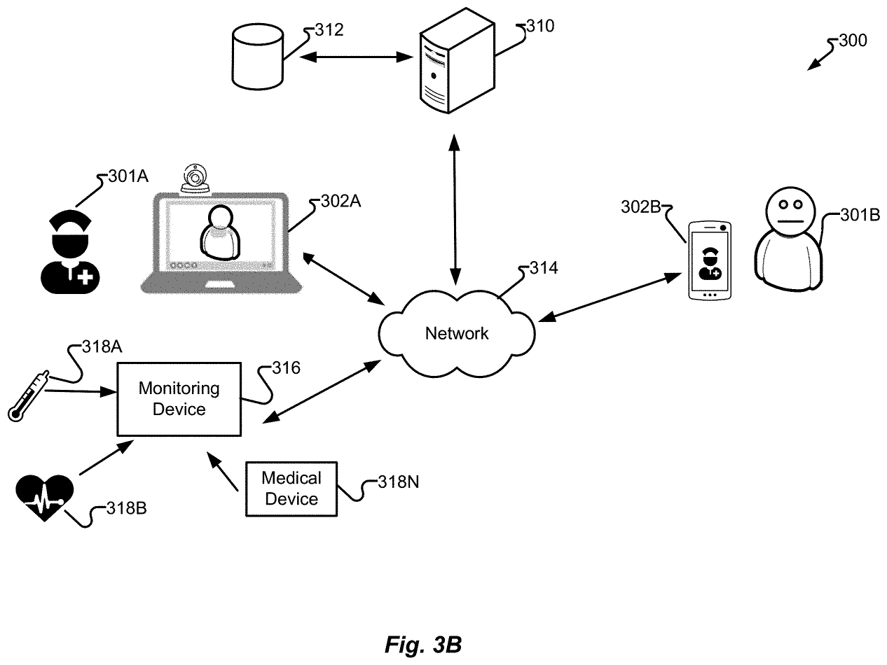

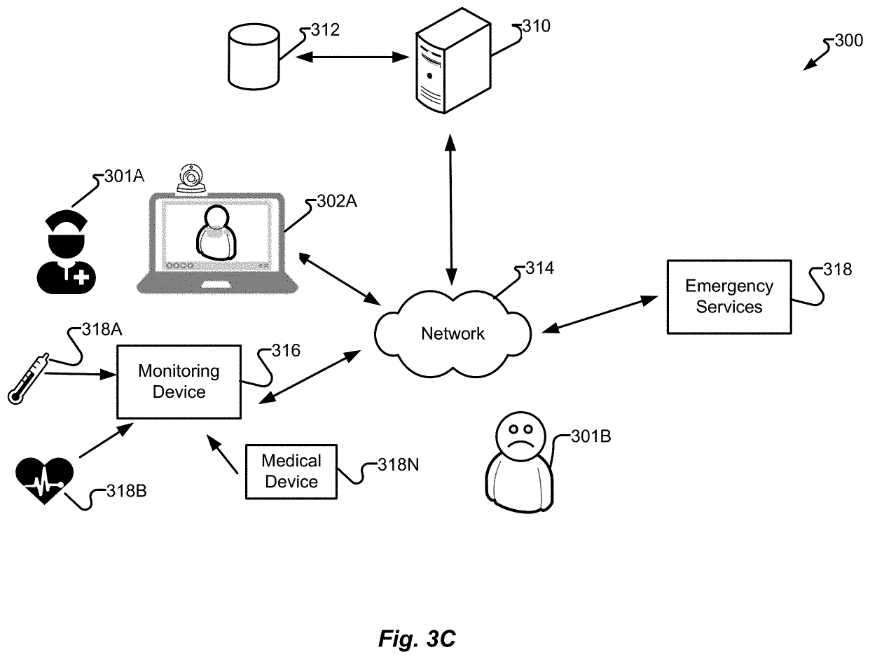

FIGS. 3A-3B depict a system 300 in accordance with embodiments of the present disclosure.

As illustrated in FIG. 3A, a medical device 318 may be a peripheral device connected to the monitoring device 316. Additionally, video/image data associated with a patient 301B indicates the patient's 301B mood is happy/comfortable. A server 310 may determine that the operation to perform is to gather additional information/verbally check-in with the patient 301B (e.g., “Is everything ok? How are you feeling?”) sent through the monitoring device 316. The monitoring device 316 may receive a verbal response for the patient 301B (e.g., “I'm fine.”). The verbal response may be received by a microphone included in the monitoring device 316. The verbal response may be processed by using NLP to determine if further action is required. The verbal response may also be transmitted with video/image data. For example, if the user 101A/101B says, “I need assistance,” a communication session may be initiated with a nurse to make further determination of the user's 101A/101B state/issue.

Referring now to FIG. 3B, a thermometer 318A and a heart rate monitor 318B are connected to the monitoring device 316. Medical devices 318N monitor vitals for the patient 301B. In additional to the medical device data (which may correspond, for example, to the medical device data 204), the server 310 receives audio/video data for the patient 301B and determines the patient 301B is experiencing low pain and requires attention. The server 310 automatically establishes a video call between a provider 301A and the patient 301B via device 302A and device 302B, respectively.

Referring now to FIG. 3C, the server 310 correlates user data 202 and medical device data 204 and determines the patient 301B requires immediate assistance (e.g., the user 301B is distressed). The server 310 dispatches emergency services 318 to a location of the patient 301B. The provider 301A may be concurrently notified of the patient 301B's state, and a video call is established.

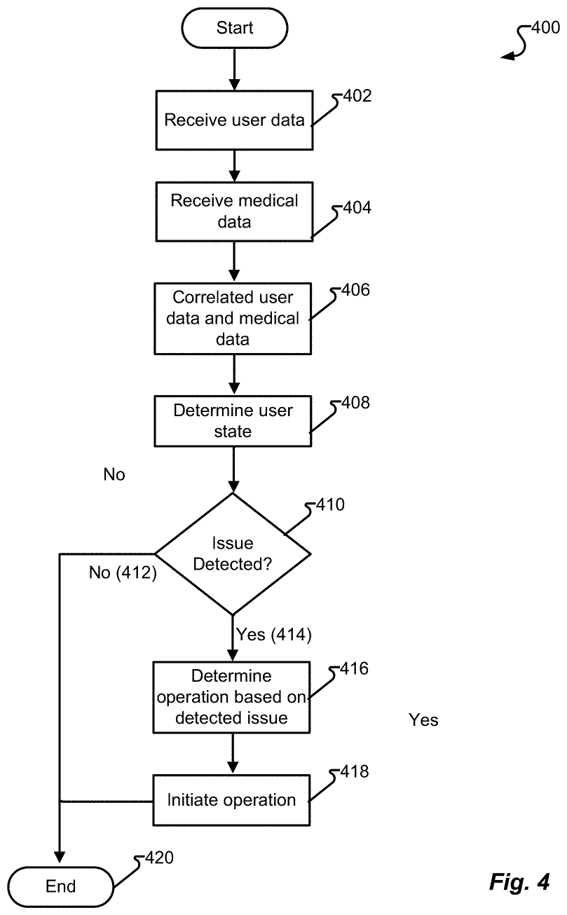

FIG. 4 depicts a process 400 in accordance with embodiments of the present disclosure. The process 400 may be embodied as one or more algorithms, encoded as machine-readable instructions that, when read by a processor, such as a processor of the server 110, cause the processor to execute the steps of the algorithm. In one embodiment, the process 400 detects a user's state and automatically performs an operation based on the user's state, which will be discussed more completely with respect to FIGS. 3A-C.

In one embodiment, the process 400 in step 402 receives user data (e.g., audio, video, location data such as the user data 202) for a user device 102A/102B and/or the monitoring device 116/316. In step 404, medical device data 204 (e.g., blood pressure, body temperature, pulse rate, respiratory rate, etc.) is received (e.g., from monitoring device 116/316 and/or medical devices 318). In step 406, the user data 202 and the medical device data 204 are correlated. For example, facial expression data may be correlated to determine a mood of the user 101A/101B. The user data 202 may be further correlated with the medical device data 204 to determine a state of the user 101A/101B. In step 408, the state of the user 101A/101B is determined (e.g., comfortable, in pain, distressed). Based on the user's 101A/101B state, the system/method determines if an issue is detected (step 410). If no issue is detected (step 412), the process 400 ends (step 420). If an issue is detected (Yes: step 414), then the process 400 proceeds to step 416 to determine the appropriate operation based on the determined state/detected issue. In step 418 the system automatically performs the determined operation, and the process 400 ends (step 420). The process 400 may continue to monitor the user data 202 or the medical device data 204 until instructions indicates that the process may end.

The steps may be performed continuously, while other steps of process 400 are executed, until the process is concluded.

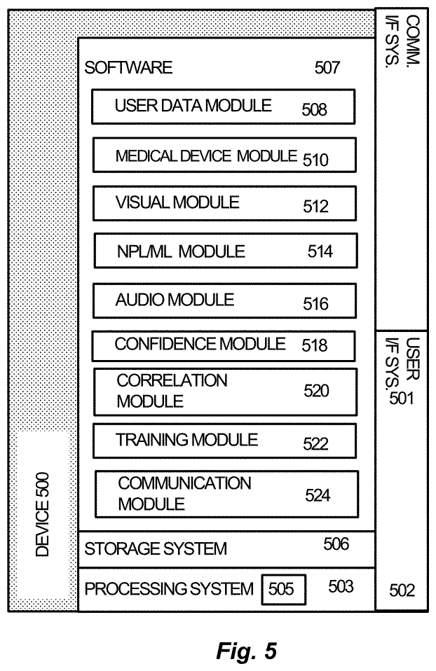

FIG. 5 depicts a device 500 in accordance with embodiments of the present disclosure. The device 500 intelligently determines the user's 101A/101B state and automatically establishes a communication session based on the determined state (e.g., text message session, audio call, video call, contact/dispatching emergency services, etc.). Similar computing systems may be included in the server 110/310, in whole or in part, described herein provide intelligent detection of the user's 101A/101B state and automatically performs an operation based on the user's 101A/101B state. The device 500 is representative of any computing system or systems with which the various operational architectures, processes, scenarios, and sequences disclosed herein for correlating user data 202 and medical device data 204 to make an intelligent determination regarding the user's 101A/101B state. In some embodiments, system comprise various components and connections to other components and/or systems.

The device 500 is an example of the server 110, although other examples may exist. The device 500 comprises a communication interface 501, a user interface module 502, and a processing system 503. The processing system 503 is linked to the communication interface 501 and the user interface module 502. The processing system 503 includes a microprocessor and/or processing circuitry 505 and a storage system 506 that stores an operating software 507. The device 500 may include other well-known components such as a battery and enclosure that are not shown for clarity. The device 500 may comprise a server, a user device, a desktop computer, a laptop computer, a tablet computing device, or some other user communication apparatus.

Communication interface 501 comprises components that communicate over communication links, such as network cards, ports, radio frequency (RF), processing circuitry 505 and software, or some other communication devices. Communication interface 501 may be configured to communicate over metallic, wireless, or optical links. Communication interface 501 may be configured to use Time Division Multiplex (TDM), Internet Protocol (IP), Ethernet, optical networking, wireless protocols, communication signaling, or some other communication format—including combinations thereof. In some implementations, communication interface 501 is configured to communicate with user devices 102, wherein the communication interface 501 is used to transfer and receive text messages, and voice and video communications for the devices. Further, the communication interface 501 may interface with a webservice, wherein the webservice may comprise medical monitoring service that can be accessed via a website.

The user interface module 502 comprises components that interact with a user 101A/101B to present media (e.g., audio/video calls) and/information (e.g., user data 202 and medical device data 204). The user interface module 502 may include a speaker, microphone, buttons, lights, display screen, touch screen, touch pad, scroll wheel, communication port, or some other user input/output apparatus—including combinations thereof. User interface module 502 may be omitted in some examples.

The processing circuitry 505 may be embodied as a single electronic microprocessor or multiprocessor device (e.g., multicore) having therein components such as control unit(s), input/output unit(s), arithmetic logic unit(s), register(s), primary memory, and/or other components that access information (e.g., data, instructions, etc.), such as received via a bus, executes instructions, and outputs data, again such as via the bus. In other embodiments, the processing circuitry 505 may comprise a shared processing device that may be utilized by other processes and/or process owners, such as in a processing array or distributed processing system (e.g., “cloud”, farm, etc.). It should be appreciated that the processing circuitry 505 is a non-transitory computing device (e.g., electronic machine comprising circuitry and connections to communicate with other components and devices). Processing circuitry 505 may operate a virtual processor, such as to process machine instructions not native to the processor (e.g., translate the Intel® 9xx chipset code to emulate a different processor's chipset or a non-native operating system, such as a VAX operating system on a Mac), however, such virtual processors are applications executed by the underlying processor and the hardware and other circuitry thereof.

The processing circuitry 505 comprises the microprocessor and other circuitry that retrieves and executes the operating software 507 from the storage system 506. The storage system 506 may include volatile and nonvolatile, removable and non-removable media implemented in any method or technology for storage of information, such as computer readable instructions, data structures, program modules, or other data. The storage system 506 may be implemented as a single storage device, but may also be implemented across multiple storage devices or sub-systems. The storage system 506 may comprise additional elements, such as a controller to read the operating software 507. Examples of storage media include random access memory, read only memory, magnetic disks, optical disks, and flash memory, as well as any combination or variation thereof, or any other type of storage media. In some implementations, the storage media may be a non-transitory storage media. In some instances, at least a portion of the storage media may be transitory. It should be understood that in no case is the storage media a propagated signal.

Processing circuitry 505 is typically mounted on a circuit board that may also hold storage system 506 and portions of the communication interface 501 and the user interface module 502. The operating software 507 comprises computer programs, firmware, or some other form of machine-readable program instructions. The operating software 507 includes a user data module 508, a medical device data module 510, a visual module 512 for intelligent video and image processing through an AI system, a Natural Language Processing (NPL)/Machine Learning (ML) module 514, an audio module 516, a confidence module 518, a correlation module 520, a training module 522, and a communication module 524, although any number of software modules within the application may provide the same operation. The operating software 507 may further include an operating system, utilities, drivers, network interfaces, applications, or some other type of software. When executed by the processing circuitry 505, the operating software 507 directs the processing system 503 to operate the device 500 as described herein.

In at least one implementation, the user data module 508, when read and executed by the processing system 503, directs the processing system 503 to receive user data 202. The user data module 508 may determine information related to the user 101A/101B (e.g., a location of the user 101A/101B, information identifying the user 101A/101B, etc.). Additionally, the user data module 508 may capture audio, image, video data associated with the user 101A/101B.

The medical device data module 510, when read and executed by the processing system 503, directs the processing system 503 to receive medical device data 204. In some embodiments, the medical device data 204 is captured by a device connected to the device 500 (e.g., a peripheral device). For example, a heartrate monitor may be connected to the device 500. In another example, a thermal camera may be included in the device 500 that is able to detect body temperature of any individuals visible to the thermal camera.

In at least one implementation, the visual module 512, when read and executed by the processing system 503, directs the processing system 503 to intelligently process video and image data using an AI driven system. In some embodiments, the video/image data is captured by the device 500 (e.g., a monitoring device 116). In other embodiments, the video/image data is received from a user device (e.g., a user device 102B). For example, a camera may capture video or image data. The video/image data may be used to analyze facial expressions of the user 101A/101B to make a determination of a mood of the user 101A/101B. In another example, the video/image data may be processed to determine a user 101A/101B has fallen, or is making irregular movements indicating assistance is required. In some examples, the visual module 512 comprises Visual Analysis and Processing module #1.

The NLP/ML module 514 when read and executed by the processing system 503, directs the processing system 503 to analyze audio data in real time to determine speech/audio characteristics (e.g., volume, intensity, range, tone, pitch, language, etc.), context, and other information. In some examples, the NPL module 514 may comprise a natural language module #2.

The audio module 516 when read and executed by the processing system 503, directs the processing system 503 to receive audio data from a user device 102. The audio data may be analyzed for audio characteristics such as keywords, intensity/loudness, pitch, tone, etc. The audio data is analyzed, preferably in real-time with other data, such as from the video analysis and processing module and/or the natural language processing module. Other data, such as the medical device data 204 may be used to make the determination that the user 101A/101B requires assistance. In some examples, the audio module 516 may comprise Audio Analysis and Processing module #3.

The confidence module 518, when read and executed by the processing system 503, directs the processing system 503 to determine a confidence score for performing an operation based on the determined state of the user 101A/101B. The confidence module 518 interfaces with the other modules, in order to determine a confidence level for the operation.

The correlation module 520, when read and executed by the processing system 503, directs the processing system 503 to correlate user data 202 and medical device data 204 to determine a state of a user 101A/101B. For example, the user data 202 may indicate that the user 101A/101B is happy (e.g., based on facial expressions) and the medical device data 204 may indicate that the user's vitals 204 are within a normal range. By correlating the user data 202 and the medical device data 204, the system determines that the user 101A/101B does not require immediate assistance, but may indicate that the provider 301A may check in later. For example, the system may send a text message to a user device 102 associated with the patient 301B that states the provider 301A will follow-up/check-in in an hour.

The training module 522, when read and executed by the processing system 503, directs the processing system 503 to user data 202, medical device data 204, and audio/video/image data to train the system/device 500 and the various modules.

The communication module 524, when read and executed by the processing system 503, directs the processing system 503 to perform the determined operation. For example, the determined operation may be to initiate a call (e.g., audio or video) between the user 101A/101B and the provider 301A (e.g., nurse, doctor, etc.). In another example, the operation may be to initiate a messaging session between the user 101A/101B and the provider 301A based on the state of the user 101A/101B. In yet another example, the operation may be to dispatch emergency services to a location of the user 101A/101B. The communication module 524 may also initiate some other alert via an alerting system.

It should be appreciated that computer readable data may be sent, received, stored, processed, and presented by a variety of components. It should also be appreciated that components illustrated may control other components, whether illustrated herein or otherwise. Ones of ordinary skill in the art will appreciate that other communication equipment may be utilized, in addition or as an alternative, to those described herein without departing from the scope of the embodiments.

In the foregoing description, for the purposes of illustration, methods were described in a particular order. It should be appreciated that in alternate embodiments, the methods may be performed in a different order than that described without departing from the scope of the embodiments. It should also be appreciated that the methods described above may be performed as algorithms executed by hardware components (e.g., circuitry) purpose-built to carry out one or more algorithms or portions thereof described herein. In another embodiment, the hardware component may comprise a general-purpose microprocessor (e.g., CPU, GPU) that is first converted to a special-purpose microprocessor. The special-purpose microprocessor then having had loaded therein encoded signals causing the, now special-purpose, microprocessor to maintain machine-readable instructions to enable the microprocessor to read and execute the machine-readable set of instructions derived from the algorithms and/or other instructions described herein. The machine-readable instructions utilized to execute the algorithm(s), or portions thereof, are not unlimited but utilize a finite set of instructions known to the microprocessor. The machine-readable instructions may be encoded in the microprocessor as signals or values in signal-producing components and included, in one or more embodiments, voltages in memory circuits, configuration of switching circuits, and/or by selective use of particular logic gate circuits. Additionally, or alternative, the machine-readable instructions may be accessible to the microprocessor and encoded in a media or device as magnetic fields, voltage values, charge values, reflective/non-reflective portions, and/or physical indicia.

In another embodiment, the microprocessor further comprises one or more of a single microprocessor, a multi-core processor, a plurality of microprocessors, a distributed processing system (e.g., array(s), blade(s), server farm(s), “cloud”, multi-purpose processor array(s), cluster(s), etc.) and/or may be co-located with a microprocessor performing other processing operations. Any one or more microprocessor may be integrated into a single processing appliance (e.g., computer, server, blade, etc.) or located entirely or in part in a discrete component connected via a communications link (e.g., bus, network, backplane, etc. or a plurality thereof).

Examples of general-purpose microprocessors may comprise, a central processing unit (CPU) with data values encoded in an instruction register (or other circuitry maintaining instructions) or data values comprising memory locations, which in turn comprise values utilized as instructions. The memory locations may further comprise a memory location that is external to the CPU. Such CPU-external components may be embodied as one or more of a field-programmable gate array (FPGA), read-only memory (ROM), programmable read-only memory (PROM), erasable programmable read-only memory (EPROM), random access memory (RAM), bus-accessible storage, network-accessible storage, etc. These machine-executable instructions may be stored on one or more machine-readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

In another embodiment, a microprocessor may be a system or collection of processing hardware components, such as a microprocessor on a client device and a microprocessor on a server, a collection of devices with their respective microprocessor, or a shared or remote processing service (e.g., “cloud” based microprocessor). A system of microprocessors may comprise task-specific allocation of processing tasks and/or shared or distributed processing tasks. In yet another embodiment, a microprocessor may execute software to provide the services to emulate a different microprocessor or microprocessors. As a result, a first microprocessor, comprised of a first set of hardware components, may virtually provide the services of a second microprocessor, whereby the hardware associated with the first microprocessor may operate using an instruction set associated with the second microprocessor.

While machine-executable instructions may be stored and executed locally to a particular machine (e.g., personal computer, mobile computing device, laptop, etc.), it should be appreciated that the storage of data and/or instructions and/or the execution of at least a portion of the instructions may be provided via connectivity to a remote data storage and/or processing device or collection of devices, commonly known as “the cloud,” but may include a public, private, dedicated, shared and/or other service bureau, computing service, and/or “server farm.” Examples of the microprocessors as described herein may include, but are not limited to, at least one of Qualcomm® Snapdragon® 800 and 801, Qualcomm® Snapdragon® 610 and 615 with 4G LTE Integration and 64-bit computing, Apple® A7 microprocessor with 64-bit architecture, Apple® M7 motion coprocessors, Samsung® Exynos® series, the Intel® Core™ family of microprocessors, the Intel® Xeon® family of microprocessors, the Intel® Atom™ family of microprocessors, the Intel Itanium® family of microprocessors, Intel® Core® i5-4670K and i7-4770K 22 nm Haswell, Intel® Core® i5-3570K 22 nm Ivy Bridge, the AMD® FX™ family of microprocessors, AMD® FX-4300, FX-6300, and FX-8350 32 nm Vishera, AMD® Kaveri microprocessors, Texas Instruments® Jacinto C6000™ automotive infotainment microprocessors, Texas Instruments® OMAP™ automotive-grade mobile microprocessors, ARM® Cortex™-M microprocessors, ARM® Cortex-A and ARIV1926EJ-S™ microprocessors, other industry-equivalent microprocessors, and may perform computational functions using any known or future-developed standard, instruction set, libraries, and/or architecture. Any of the steps, functions, and operations discussed herein can be performed continuously and automatically.

The exemplary systems and methods of the present disclosure have been described in relation to communications systems and components and methods for monitoring, enhancing, and embellishing communications and messages. However, to avoid unnecessarily obscuring the present disclosure, the preceding description omits a number of known structures and devices. This omission is not to be construed as a limitation of the scope of the present disclosure. Specific details are set forth to provide an understanding of the present disclosure. It should, however, be appreciated that the present disclosure may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components or portions thereof (e.g., microprocessors, memory/storage, interfaces, etc.) of the system can be combined into one or more devices, such as a server, servers, computer, computing device, terminal, “cloud” or other distributed processing, or collocated on a particular node of a distributed network, such as an analog and/or digital telecommunications network, a packet-switched network, or a circuit-switched network. In another embodiment, the components may be physical or logically distributed across a plurality of components (e.g., a microprocessor may comprise a first microprocessor on one component and a second microprocessor on another component, each performing a portion of a shared task and/or an allocated task). It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

Also, while the flowcharts have been discussed and illustrated in relation to a particular sequence of events, it should be appreciated that changes, additions, and omissions to this sequence can occur without materially affecting the operation of the present disclosure.

A number of variations and modifications of the present disclosure can be used. It would be possible to provide for some features of the present disclosure without providing others.

In yet another embodiment, the systems and methods of the present disclosure can be implemented in conjunction with a special purpose computer, a programmed microprocessor or microcontroller and peripheral integrated circuit element(s), an ASIC or other integrated circuit, a digital signal microprocessor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of the present disclosure. Exemplary hardware that can be used for the present disclosure includes computers, handheld devices, telephones (e.g., cellular, Internet enabled, digital, analog, hybrids, and others), and other hardware known in the art. Some of these devices include microprocessors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein as provided by one or more processing components.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with the present disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of the present disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Embodiments herein comprising software are executed, or stored for subsequent execution, by one or more microprocessors and are executed as executable code. The executable code being selected to execute instructions that comprise the particular embodiment. The instructions executed being a constrained set of instructions selected from the discrete set of native instructions understood by the microprocessor and, prior to execution, committed to microprocessor-accessible memory. In another embodiment, human-readable “source code” software, prior to execution by one or more microprocessors, is first converted to system software to comprise a platform (e.g., computer, microprocessor, database, etc.) specific set of instructions selected from the platform's native instruction set.

Although the present disclosure describes components and functions implemented in the embodiments with reference to particular standards and protocols, the present disclosure is not limited to such standards and protocols. Other similar standards and protocols not mentioned herein are in existence and are considered to be included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, sub combinations, and subsets thereof. Those of skill in the art will understand how to make and use the present disclosure after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and\or reducing cost of implementation.

The foregoing discussion of the present disclosure has been presented for purposes of illustration and description. The foregoing is not intended to limit the present disclosure to the form or forms disclosed herein. In the foregoing Detailed Description, for example, various features of the present disclosure are grouped together in one or more embodiments, configurations, or aspects for the purpose of streamlining the disclosure. The features of the embodiments, configurations, or aspects of the present disclosure may be combined in alternate embodiments, configurations, or aspects other than those discussed above. This method of disclosure is not to be interpreted as reflecting an embodiment that the present disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the present disclosure.

Moreover, though the description of the present disclosure has included description of one or more embodiments, configurations, or aspects and certain variations and modifications, other variations, combinations, and modifications are within the scope of the present disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

What is claimed is:

1. A device, the device comprising:
a network interface to a network;
a storage component comprising a non-transitory storage device; and
a processor, comprising at least one microprocessor;
wherein machine-executable instructions, upon being accessed by the processor, cause the processor to:
receive user data associated with a user;
receive medical device data associated with the user;
correlate the user data and the medical device data to determine a state of the user;
determine an operation based on the determined state of the user; and
automatically initiate the determined operation.

2. The device of claim 1, wherein the user data comprises at least one of: facial expression data, positioning data, movement data, and/or mood data.

3. The device of claim 2, wherein the user data is received via a microphone and/or a camera of a user device associated with the user.

4. The device of claim 1, wherein the medical device data comprises at least one of: heart rate, pulse rate, body temperature, respiratory rate, and/or blood pressure.

5. The device of claim 1, wherein the operation comprises a text message to another user.

6. The device of claim 1, wherein the operation comprises an audio call to another user.

7. The device of claim 1, wherein the operation comprises a video call to another user.

8. A method, the method comprising:
receiving user data associated with a user;
receiving medical device data associated with the user;
correlating the user data and the medical device data to determine a state of the user;
determining an operation based on the determined state of the user; and
automatically initiating the determined operation.

9. The method of claim 8, wherein the user data comprises at least one of: facial expression data, positioning data, movement data, and/or mood data.

10. The method of claim 9, wherein the user data is received via a microphone and/or a camera of a user device associated with the user.

11. The method of claim 8, wherein the medical device data comprises at least one of: heart rate, pulse rate, body temperature, respiratory rate, and blood pressure.

12. The method of claim 8, wherein the operation comprises one of: a text message, an audio call, or a video call to another user.

13. The method of claim 8, wherein the operation comprises dispatching emergency services to a location of the user.

14. The method of claim 8, wherein the state of the user comprises one of: neutral, happy, or distressed.

15. A non-transitory, computer-readable medium comprising a set of instructions stored therein which, when executed by a processor, causes the processor to:
receive user data associated with a user;
receive medical device data associated with the user;
correlate the user data and the medical device data to determine a state of the user;
determine an operation based on the determined state of the user; and
automatically initiate the determined operation.

16. The non-transitory, computer-readable medium of claim 15, wherein the user data comprises at least one of: facial expression data, positioning data, movement data, and/or mood data, and wherein the user data is received via a microphone and/or a camera of a user device associated with the user.

17. The non-transitory, computer-readable medium of claim 15, wherein the medical device data comprises at least one of: heart rate, pulse rate, body temperature, respiratory rate, and blood pressure.

18. The non-transitory, computer-readable medium of claim 15, wherein the operation comprises a text message to another user.

19. The non-transitory, computer-readable medium of claim 15, wherein the operation comprises an audio call to another user.

20. The non-transitory, computer-readable medium of claim 15, wherein the operation comprises dispatching emergency services to a location of the user.
